(19) United States
(12) Patent Application Publication
(10) Pub. No.: US 2004/0157772 A1
(43) Pub. Date: Aug. 12, 2004

(54) Use of a gatran for the manufacture of a medicament of the treatment of pulmonary fibrosis

(76) Inventor: Ian Kirk, Loughborough (GB)

Correspondence Address: ROPES & GRAY LLP, ONE INTERNATIONAL PLACE, BOSTON, MA, 02110-2624, US

(21) Appl. No.: 10/477,775

(22) PCT Filed: May 16, 2002
(86) PCT No.: PCT/SE02/00950

(30) Foreign Application Priority Data
May 18, 2001 (SE) 0101762-3

(51) Int. Cl. A61K 38/17

(52) U.S. Cl. 514/12

(57) ABSTRACT

According to the invention there is provided the use of a gatran, such as inogatran or melagatran, or a pharmaceutically-acceptable derivative thereof, for the manufacture of a medicament for the treatment of pulmonary fibrosis.

This invention relates to a new use of certain low molecular weight thrombin inhibitors.

Interstitial lung disease (ILD) is a general term that includes chronic lung disorders, often characterised, initially, by inflammation of various parts of the lung, including the bronchioles, the capillaries and, particularly, the alveoli.

Such inflammation may lead to damage, in particular scarring (fibrosis) of various parts of the lung, including the alveoli and in the interstitium, and/or regions of severe thickening of the alveolar walls. When such scarring and/or thickening occur, a chronic stiffness in the lungs and a decreased ability of the lung tissue to transport oxygen often results. Such histological changes in the lung tissue are typically referred to as pulmonary fibrosis (PF).

Although the course of PF is unpredictable, patients may experience a variety of symptoms including dyspnea and a dry cough, which is often ignored at first. As PF progresses, dyspnea becomes a major problem, leading to severe difficulty in performing anything physical, including day-to-day tasks such as walking short distances (especially up stairs), dressing and even eating. In the later stages of disease, patients may become less able to fight infection, may need to breath oxygen continuously, and may experience hypoxemia, pulmonary hypertension, cardiac failure, ischemic attack, pulmonary embolism, stroke or infection brought on by the disease, one or more of which usually results in death.

PF may result from several known causes. These include exposure to substances that may damage/irritate the lungs, such as occupational and/or environmental exposure to e.g. dusts and fibres (such as those of metals, silica and asbestos); organic matter, which may lead to an allergic reaction (e.g. Farmer's Lung); or chemicals, including certain drugs (e.g. chemotherapeutic drugs useful in the treatment of cancer). Further, radiation therapy for e.g. breast cancer may lead to PF.

Moreover, PF may be a feature of diseases such as inter alia sarcoidosis, rheumatoid arthritis, systemic sclerosis, scleroderma, extrinsic allergic alveolitis, severe asthma, systemic granulomatosis vasculitis and adult respiratory distress syndrome (ARDS).

When the cause of PF is unknown, the disease is termed “idiopathic” PF (IPF). IPF, which is also often referred to as cryptogenic fibrosing alveolitis (CFA), is a progressive interstitial lung disease of unknown etiology. Now recognised as a distinct clinical disorder, IPF is characterised by a fibroproliferative response with only minor signs of inflammation (unlike other forms of PF, brought on, for example, by other causes listed above) and almost always causes rapid fibrotic destruction of the lung (see inter alia, Am. J. Respir. Crit. Care Med., 157 1301 (1998), ibid., 161, 646 (2000), Thorax, 51, 711 (1996) and N. Engl. J. Med., 341, 1264 (1999)).

Current thinking centres around IPF being triggered by an autoimmune disorder, in which the body's immune system attacks its own tissues, or by the after-effects of infection by e.g. a virus, or cigarette smoking.

The diagnosis and management of patients with IPF poses significant challenges. Treatments for IPF include oxygen and exercise therapies. More drastic treatments include fill lung transplantation.

Current front-line pharmaceutical treatments for IPF aim to reduce inflammation and thus arrest abnormal processes that may lead to fibrosis. Thus, at present, corticosteroids, such as prednisone, are employed. However, perhaps in view of the fact that there is no recognisably significant inflammatory component to IPF, the degree of success of these drugs in the treatment of IPF is variable at best. This is in addition to the well-documented side-effects of such treatments. Other drugs, such as immunosupressants (e.g. cyclophosphamide (cytoxan), azathioprine, colchicine, methotrexate, penicillamine and cyclosporin) have been employed, though such treatments are also known to exhibit side effects, which, in some cases, can be serious.

Thus, there is a need for alternative and/or better treatments for use in patients with, or at risk of, PF and especially IPF.

Gray et al (see Thorax (1999) 54, Abstract S62 and American Journal of Respiratory and Critical Care Medicine (1999) 159, A73) have reported that a direct thrombin inhibitor (code name UK-156406) has the ability to block collagen deposition in bleomycin-induced PF in rats. Continuous infusion of the inhibitor (0.5 mg/kg body weight/hour) via an osmotic minipump was found to reduce the stimulation of total lung collagen of bleomycin treated animals by ca 38% after 14 days.

International patent application WO 93/11152 discloses a group of compounds, including HOOC—CH₂-(R)Cha-Pic-Nag-H (wherein Cha represents cyclohexylalaninyl, Pic represents (S)-pipecolinic acyl and Nag represents noragmatino), which is also known as inogatran (see Example 67 of WO 93/11152, and the list of abbreviations in that document), which are useful as thrombin inhibitors and thus as anticoagulants. The use of these compounds in the treatment of PF is not mentioned.

International patent application WO 94/29336 discloses a group of compounds that are useful as inhibitors of serine proteases, such as thrombin and/or kininogenases. The thrombin-inhibiting compounds are thus indicated as anticoagulants, and the kininogenase-inhibiting compounds as anti-inflammatory agents. Again, PF is not mentioned.

One of the thrombin-inhibiting compounds that is specifically disclosed in WO 94/29336 is HOOC—CH₂-(R)Cgl-Aze-Pab-H (wherein Cgl represents cyclohexylglycinyl, Aze represents (S)-azetidine-2-carboxyl, and Pab represents para-amidinobenzylamino), which is also known as melagatran (see Example 1 of WO 94/29336, and the list of abbreviations in that document). International Patent Application WO 97/23499 discloses prodrugs of inter alia melagatran. One of the many indications mentioned in WO 97/23499 is PF following treatment with radiation or chemotherapy. IPF is neither mentioned nor suggested.

We have now found that the gatrans and derivatives thereof prevent collagen deposition in the lung and may thus be used in the treatment of PF, such as IPF.

According to a first aspect of the invention there is provided the use of a gatran, or a pharmaceutically-acceptable derivative thereof, for the manufacture of a medicament for the treatment of PF.

Preferably, when the pharmaceutically-acceptable derivative is a prodrug of melagatran, and, more preferably, when the gatran itself is melagatran, then the disease to be treated is IPF.

The term “PF” will be understood by those skilled in the art to include any condition characterised by one or more of (a) collagen deposition in the lung, (b) scarring (fibrosis) of the lung (including the alveoli and in the interstitium), and/or (c) regions of severe thickening of the alveolar walls, one or more of which may result in a chronic stiffness in the lungs and/or a decreased ability of the lung tissue to transport oxygen.

According to a second aspect of the invention there is provided the use of a gatran, or a pharmaceutically-acceptable derivative thereof, for the manufacture of a medicament for the prevention of collagen deposition, and/or the treatment of a disease characterised thereby, e.g. in the lung.

The PF may be a secondary fibrosis, which may be brought on by an inflammatory condition, such as sarcoidosis, rheumatoid arthritis, systemic sclerosis, scleroderma, extrinsic allergic alveolitis, severe asthma, systemic granulomatosis vasculitis and/or adult respiratory distress syndrome (ARDS), or it may be IPF.

The term “IPF” will be understood to include any form of PF where the underlying causes of the condition are unknown and/or to include the definition provided in the consensus statement in Am. J Respir. Crit. Care Med., 161, 646 (2000), the relevant disclosure in which document is hereby incorporated by reference.

Particular forms of IPF that may be mentioned include inter alia desquamative interstitial pneumonitis (DIP), acute interstitial pneumonia (AIP), non-specific interstitial pneumonia (NSIP), respiratory bronchiolitis-associated interstitial lung disease (RBILD), bronchiolitis obliterans organising pneumonia (BOOP), lymphoid interstitital pneumonia (LIP) and, particularly, usual interstitial pneumonitis (UIP) (see, for example, Am. J. Respit. Crit. Care Med., 157, 1301 (1998)).

Treatment of PF includes therapeutic treatment as well as prophylactic treatment. By prophylactic treatment, we include prevention (inhibition) of the progress of PF in patients that have the disease.

Preferred disease states include IPF.

The term “gatran” will be understood to include inogatran and melagatran. Preferred gatrans include inogatran.

“Pharmaceutically-acceptable derivatives” of gatrans include salts (e.g. pharmaceutically-acceptable non-toxic organic or inorganic acid addition salts) and solvates. It will be appreciated that the term further includes derivatives that have the same biological function and/or activity as the relevant gatran. Moreover, for the purposes of this invention, the term also includes prodrugs of the relevant gatran. The term “prodrug” includes any composition of matter that, following oral or parenteral administration, is metabolised in vivo to form the relevant gatran in an experimentally-detectable amount, and within a predetermined time (e.g. within a dosing interval of between 6 and 24 hours (i.e. once to four times daily)). For the avoidance of doubt, the term “parenteral” administration includes all forms of administration other than oral administration. Prodrugs of melagatran that may be mentioned include those disclosed generically and specifically in international patent application WO 97/23499. Preferred prodrugs are those of the formula R¹O₂C—CH₂-(R)Cgl-Aze-Pab-OH (see the list of abbreviations in WO 97/23499 and above), wherein R¹ represents C₁₋₁₀ alkyl or benzyl, such as linear or branched C₁₋₆ alkyl (e.g. C₁₋₄ alkyl, especially methyl, n-propyl, i-propyl, t-butyl and, particularly, ethyl) and the OH group replaces one of the amidino hydrogens in Pab.

Gatrans, and derivatives thereof, may be administered for systemic delivery using appropriate means of administration that are known to the skilled person.

Thus, in accordance with the invention, gatrans, and derivatives thereof, may be administered orally, intravenously, subcutaneously, buccally, is rectally, dermally, nasally, tracheally, bronchially, topically, by any other parenteral route, or, particularly via inhalation, especially to the lung, in the form of a pharmaceutical preparation comprising the active ingredient in a pharmaceutically-acceptable dosage form. Depending on the disorder, and the patient, to be treated, as well as the route of administration, the compositions may be administered at varying doses.

Preferred modes of delivery are systemic. For the gatrans themselves, preferred modes of administration are parenteral, more preferably intravenous, subcutaneous or by inhalation. For prodrugs of the gatrans, especially melagatran, preferred modes of administration are oral, intravenous, subcutaneous or by inhalation.

In the therapeutic treatment of mammals, and especially humans, gatrans and derivatives thereof may be administered alone, but will generally be administered as a pharmaceutical formulation in admixture with a pharmaceutically-acceptable adjuvant, diluent or carrier, which may be selected with due regard to the intended route of administration and standard pharmaceutical practice.

Suitable formulations for use in administering inogatran and derivatives thereof are described in the literature, for example as described in inter alia international patent applications WO 93/11152, WO 96/14084, WO 99/27912, WO 99/27913 and WO 00/76504, the disclosures in which documents are hereby incorporated by reference.

Suitable formulations for use in administering melagatran and derivatives (including prodrugs) thereof are described in the literature, for example as described in inter alia international patent applications WO 94/29336, WO 96/14084, WO 96/16671, WO 97/23499, WO 97/39770, WO 97/45138, WO 98/16252, WO 99/27912, WO 99/27913, WO 00/12043 and WO 00/13671, the disclosures in which documents are hereby incorporated by reference.

Otherwise, the preparation of suitable formulations, for example for administration of active ingredient by inhalation, may be achieved non-inventively by the skilled person using routine techniques (see, for example, Inhalation Aerosols: Physiological and Biological Basis for Therapy (ed. Anthony J. Hickey), Lung Biology in Health and Disease, Volume 94. Marcel Dekker Inc. (1996) and Respiratory Drug Delivery (ed. Peter R. Byron), CRC Press Inc. (1990)).

The amount of gatran or derivative in the formulation will depend on the severity of the condition, and on the patient, to be treated, as well as the compound(s) which is/are employed, but may be determined non-inventively by the skilled person.

According to a further aspect of the invention there is provided a pharmaceutical formulation for use in the treatment of PF comprising an effective amount of a gatran, or a pharmaceutically-acceptable derivative thereof, in admixture with a pharmaceutically-acceptable adjuvant, diluent or carrier.

In the treatment of PF, gatrans and derivatives (including prodrugs) thereof may also be combined with other agents known for use in the treatment of PF, for example corticosteroids, such as prednisone, immunosuppressant drugs including cyclophosphamide (cytoxan), azathioprine, colchicine, methotrexate, penicillamine, cyclosporin and interferon gamma, and/or anti-fibrotic agents such as pirfenidone.

When gatrans, and derivatives thereof, are “combined” with other therapeutic agents in this way, the active ingredients may be administered together in the same formulation, or administered separately (simultaneously or sequentially) in different formulations.

Suitable doses of gatrans and derivatives thereof, in the therapeutic and/or prophylactic treatment of mammalian, especially human, patients may be determined routinely by the medical practitioner or other skilled person, and include the respective doses discussed in the prior art documents mentioned hereinbefore, the disclosures in which documents are hereby incorporated by reference.

For example, suitable doses of inogatran (when inhaled) and melagatran (when administered intravenously, subcutaneously or by inhalation), and prodrugs and derivatives of either, in the therapeutic and/or prophylactic treatment of mammalian, especially human, patients include those which give a mean plasma concentration of active compound of up to 10 μmol/L, for example in the range 0.001 to 5 μmol/L (e.g. 0.01 to 1 μmol/L, such as 0.05 to 0.5 μmol/L) over the course of treatment of the relevant condition.

In any event, the physician, or the skilled person, will be able to determine the actual dosage which will be most suitable for an individual patient, which is likely to vary with the condition that is to be treated, as well as the age, weight, sex and response of the particular patient to be treated. The above-mentioned dosages are exemplary of the average case; there can, of course, be individual instances where higher or lower dosage ranges are merited, and such are within the scope of this invention.

The skilled person will also appreciate that a gatran, or a derivative thereof, may be administered in an appropriate dose on an “as required” basis (i.e. as needed or desired).

According to a further aspect of the invention there is provided a method of preventing or treating PF, which comprises administering a therapeutically-effective amount of a gatran, or a pharmaceutically-acceptable derivative thereof, to a patient in need of such treatment.

The use and method described herein may have the advantage that, in the treatment of PF, and especially IPF, gatrans and derivatives thereof may not possess disadvantages of known therapies. The use and method described herein may also have the advantage that gatrans and derivatives thereof may be more efficacious than, be less toxic than, have a broader range of activity than, be more potent than, produce fewer side effects than, be more easily absorbed than, or that they may have other useful pharmacological properties over, compounds known in the prior art for the treatment of PF, such as IPF.

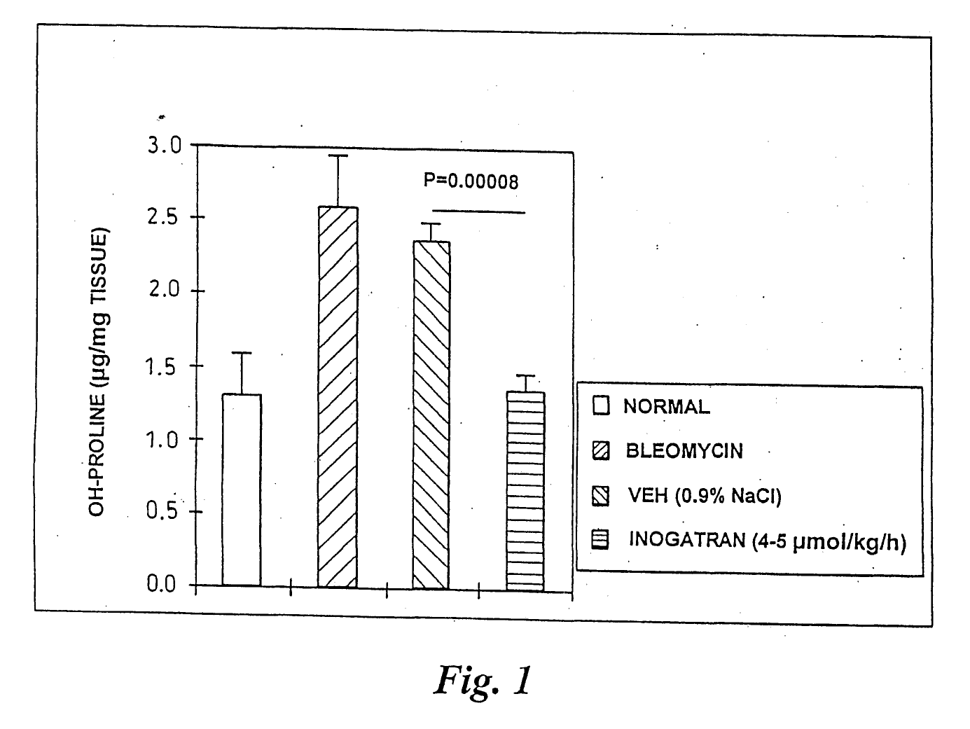

The invention is illustrated, but in no way limited, by the following example, in which FIG. 1 shows the total collagen deposition in the lungs at the end of a bleomycin-induced lung fibrosis study for four study groups of rats.

### EXAMPLE 1

### Evaluation of Inogatran in a Bleomycin-Induced Lung Fibrosis Model

Forty male Sprague Dawley rats weighing 200 g were kept in cages (2 rats/cage) covered with filter tops to prevent spreading of carcinogenic metabolites of bleomycin. The rats had free access to food and water.

The rats were split into four groups:

Group 1—8 rats—received 0.9% NaCl (1 mL/kg) as a control (only 6 animals were ultimately used, the other 2 were used for different purposes).

Group 2—8 rats—received induction by way of an i.t. 2.5 mg/kg (1 mL/kg) bleomycin instillation (a 15 IE injection solution (Lundbeck)).

Group 3—12 rats—received bleomycin induction as above, plus a constant i.v. infusion of 0.9% NaCl solution (5.0 μL/h) (only 8 animals were ultimately used, the other 4 were used for different purposes).

Group 4—12 rats—received bleomycin induction as above, plus a constant i.v. infusion of inogatran (5 μmol/kg/h) loaded in Alzet® 2ML2 pumps at a concentration of 200 μmol/mL (88 mg/mL) in 0.9% NaCl (only 8 animals were ultimately used, the other 4 were used for different purposes).

The rats were anaesthetised by way of an i.p. injection (2 mL/kg) of Ketalar™ (50 mg/mL ketamin; Parke-Davis)/Rompun.vet™ (20 mg/mL xylazin; Bayer) (1.75 mL/0.35 mL). A pre-filled minipump connected to a PE60 catheter was inserted into the jugularis vein. A s.c. pocket was created on the back of the animal. The pump was led into this pocket. The incision on the neck was closed with wound clips.

The pre-filled pump with catheter was incubated in 37° C. 0.9% NaCl overnight before implantation to enable immediate pumping after surgery. This was to avoid clotting in the catheter.

Plasma and bronchiolar lavage fluid (BAL) were collected from four rats in groups 3 and 4, and from two rats in group 1, six days after induction, for quantification of plasma concentration of compound and thrombin activity in BAL.

After 14 days, all rats received an overdose of pentobarbital (i.p.). The rats were weighed and the lung was dissected and weighed. One lobe was placed in Histofix™ (buffered 5% formaldehyde solution) for histological examination. The remaining lungs were snap frozen in liquid nitrogen and stored prior to analysis at −70° C.

The following parameters were measured:

Body weight (body weight gained during the experiment).

Lung weight (total lung wet-weight).

Total collagen (quantification of hydroxyproline according to the procedure described in Stegemann-Stadler et al, Determinations of Hydroxyproline, Clin. Chim. Acta (1967) 18, 267-273).

BAL (thrombin activity in BAL on day 6).

Concentration of inogatran in plasma on day 6.

The analysis of inogatran plasma on day 6 after implantation indicated that the pumps delivered the compound. Untreated rats (2) and rats receiving vehicle from the pump (4) had undetectable levels of inogatran in plasma, while the rats receiving compound had a plasma concentration in the range 2 to 7 μmol/L (approximately).

Rats instilled with bleomycin shows a significantly slower body weight gain (BWG) compared to the controls.

Bleomycin instillation resulted in a significant increase in total lung weight from approximately 1.5-2.5 g/lung in all groups. Inogatran (4-5 μmol/kg/hr) showed no effect on total lung weight (wet-weight).

Analysis of hydroxyproline content (μg/mL tissue) in the lungs showed a two-fold increase in the bleomycin group compared to normal controls (2.59±0.27 μg/mg tissue, compared to 1.31±0.27 μg/mg tissue, see also FIG. 1). Inogatran treatment showed an almost complete inhibition of this collagen deposition (1.37±0.10 μg/mg tissue, compared to its control value of 2.34±0.14 μg/mg tissue, P=0.0008) at day 14.

In summary, this study showed expected changes in body weight, lung weight and hydroxyproline content in the bleomycin control compared to normal rats. However, there was almost complete inhibition of collagen deposition as a result of treatment with inogatran (cf. Gray et al supra).

These data demonstrate the potential utility of gatran compounds in the treatment of PF.

1. The use of a gatran, or a pharmaceutically-acceptable derivative thereof, for the manufacture of a medicament for the treatment of pulmonary fibrosis.

2. The use of a gatran, or a pharmaceutically-acceptable derivative thereof, for the manufacture of a medicament for the prevention of collagen deposition in the lung.

3. A method of treatment of pulmonary fibrosis, which comprises administering a therapeutically effective amount of a gatran, or a pharmaceutically-acceptable derivative thereof, to a patient in need of such treatment.

4. A pharmaceutical formulation for use in the treatment of pulmonary fibrosis, which formulation comprises an effective amount of a gatran, or a pharmaceutically-acceptable derivative thereof.

5. Use of a gatran, or a pharmaceutically-acceptable derivative thereof, for the treatment of pulmonary fibrosis, by administering a gatran, or a pharmaceutically-acceptable derivative thereof, to a patient.

6. The use of a gatran, or a pharmaceutically-acceptable derivative thereof, in the treatment of pulmonary fibrosis.

7. Use, method or formulation as claimed in any one of claims 1 to 6 (as appropriate), wherein the gatran is inogatran.

8. Use, method or formulation as claimed in any one of claims 1 to 6 (as appropriate), wherein the gatran is melagatran.

9. Use, method or formulation as claimed in claim 8, wherein the derivative of melagatran is a prodrug of melagatran.

10. Use, method or formulation as claimed in claim 9, wherein the prodrug is of the formula
R¹O₂C—CH₂-(R)Cgl-Aze-Pab-OH,
wherein R¹ represents linear or branched C₁₋₆ alkyl and the OH group replaces one of the amidino hydrogens in Pab.

11. Use, method or formulation as claimed in claim 10, wherein R¹ represents methyl, ethyl, n-propyl, i-propyl or t-butyl.

12. Use, method or formulation as claimed in any one of claims 1 or 3 to 11, wherein the pulmonary fibrosis is a secondary fibrosis.

13. Use, method or formulation as claimed in claim 12, wherein the fibrosis is brought on by an inflammatory condition.

14. Use, method or formulation as claimed in claim 13, wherein the condition is sarcoidosis, rheumatoid arthritis, systemic sclerosis, scleroderma, extrinsic allergic alveolitis, severe asthma, systemic granulomatosis vasculitis or adult respiratory distress syndrome.

15. Use, method or formulation as claimed in any one of claims 1 or 3 to 11, wherein the pulmonary fibrosis is idiopathic pulmonary fibrosis.
